(19) United States
(12) Patent Application Publication
(10) Pub. No.: US 2014/0025087 A1
(43) Pub. Date: Jan. 23, 2014

(54) INVAGINATING VALVULOPLASTY BALLOON CATHETER AND METHODS OF USE THEREFOR

(71) Applicant: BOSTON SCIENTIFIC SCIMED, INC., Maple Grove, MN (US)

(72) Inventor: Leonard B. Richardson, Minneapolis, MN (US)

(21) Appl. No.: 13/944,641

(22) Filed: Jul. 17, 2013

Related U.S. Application Data
(60) Provisional application No. 61/672,623, filed on Jul. 17, 2012.

(51) Int. Cl. A61M 29/02

(52) U.S. Cl. CPC A61M 29/02
USPC 606/127

(57) ABSTRACT

The disclosure pertains to an invaginating inflatable balloon catheter and methods of using the catheter. The catheter of the disclosure includes an outer tubular body, an inner tubular body, and an inflatable balloon attached to the tubular bodies such that the balloon is adapted to be inflated within a valve or other tissue to be dilated. During use, the dilating balloon is slowly deflated as aspiration is applied to the inner tubular body and the inflatable balloon is withdrawn into the outer tubular body by proximal motion of the inner tubular body.

### RELATED APPLICATIONS

This application claims priority to U.S. Provisional Application No. 61/672,623 filed Jul. 17, 2012.

### BACKGROUND

Valve stenosis or calcification is a frequent expression of valvular heart disease, and may often be a leading indicator for balloon valvuloplasty and/or valve replacement therapy in Europe and the United States. The prevalence of valve stenosis tends to increase in older population groups. In such population groups, balloon valvuloplasty may be beneficial in improving the lifestyle of patients suffering from valve stenosis and may also form a useful component of a valve replacement procedure.

A continuing need exists for improved balloon valvuloplasty devices and methods as an alternative to traditional valve replacement surgery or as an adjunct thereto.

### SUMMARY

This disclosure pertains to an invaginating valvuloplasty balloon catheter comprising an outer tubular body having a distal end and a proximal end; an inner tubular body having a distal end and a proximal end defining a lumen therebetween adapted to receive guidewire and together with the outer tubular body defining an annular inflation lumen therebetween; and an inflatable balloon element having a distal end, a proximal end an uninflated state and an inflated state, wherein the distal end of the inflatable balloon element is sealingly attached to distal end of the inner tubular body and the proximal end of the inflatable balloon element is sealingly attached to the outer body; further wherein the outer body is sized and adapted to contain the inflatable balloon element in an invaginated configuration while maintaining a fluid connection between a source of inflation medium at the proximal end thereof and the proximal end of the inner tubular body is adapted to connect with a source of aspiration.

This disclosure further pertains to a method of performing a valvuloplasty comprising inserting a guidewire through a patient's vasculature; inserting the guidewire through the valve to be dilated; advancing an invaginating valvuloplasty balloon catheter through the patient's vasculature; advancing a balloon element of an invaginating valvuloplasty balloon catheter at least partially through the valve to be dilated; inflating the balloon element of the invaginating valvuloplasty balloon catheter by introducing an inflation medium through a proximal inflation port associated with an outer tubular body of the invaginating valvuloplasty balloon catheter thereby at least partially inflating the balloon and dilating the valve to be dilated; removing the guidewire from the invaginating valvuloplasty balloon catheter; applying aspiration to an aspiration lumen associated with inner tubular body while partially removing at least a portion of the inflation medium from the balloon element and withdrawing the inner tubular body proximally through the outer tubular body thereby removing debris dislodged by dilation of the valve to be dilated; deflating the balloon element by removing the inflation medium; and removing the invaginating valvuloplasty balloon catheter from the patient's body.

### BRIEF DESCRIPTION OF DRAWINGS

FIG. 1 illustrates schematically an inflated invaginating valvuloplasty balloon catheter of the disclosure.

FIG. 2 provides a cross-sectional view of an invaginating valvuloplasty balloon catheter of FIG. 1.

FIG. 3 illustrates placement of a guidewire in preparation for a valvuloplasty procedure.

FIG. 4 illustrates the advance of an invaginating valvuloplasty balloon catheter over a guidewire.

FIG. 5 illustrates initial placement of an invaginating valvuloplasty balloon catheter within a valve.

FIG. 6 illustrates partial inflation of the balloon of an invaginating valvuloplasty balloon catheter within a valve.

FIG. 7 illustrates inflation of the balloon of an invaginating valvuloplasty balloon catheter within a valve.

FIG. 8 illustrates an inflated invaginating valvuloplasty balloon catheter with the guidewire removed.

FIG. 9 illustrates in cross-section an inflated invaginating valvuloplasty balloon catheter dilating a valve and associated plaque.

FIG. 10 illustrated in cross-section a partially inflated invaginating valvuloplasty balloon catheter as it partially invaginates with aspiration removing debris.

FIG. 11 illustrated in cross-section a nearly deflated invaginating valvuloplasty balloon catheter as it continues to invaginate with aspiration removing debris.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary.

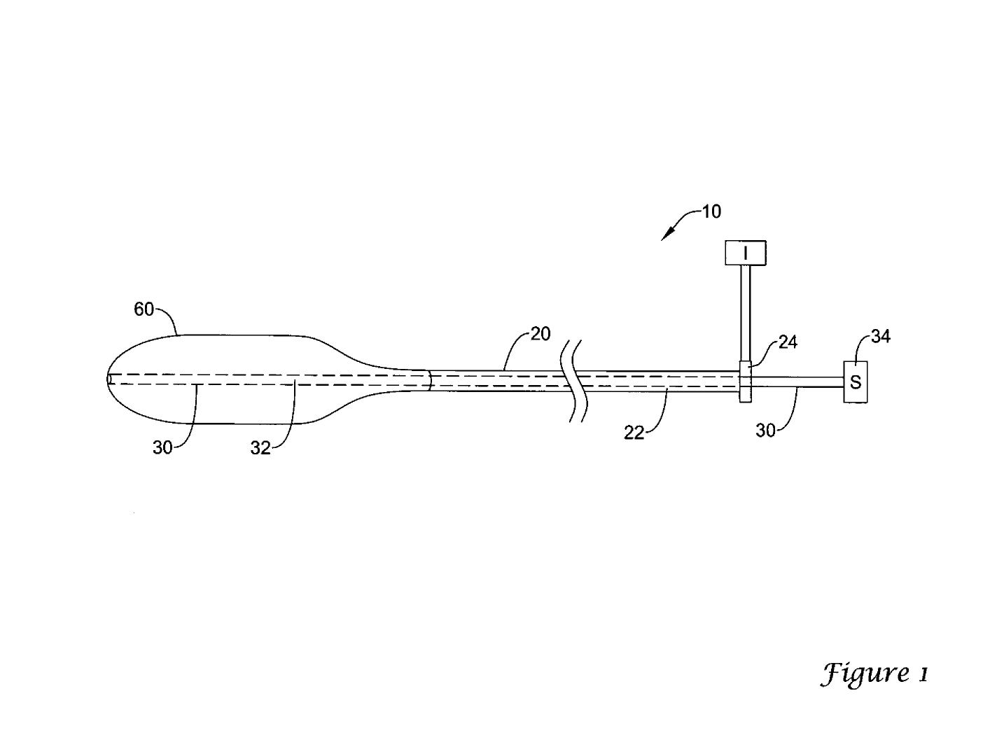

FIG. 1, illustrates somewhat schematically an invaginating valvuloplasty balloon catheter 10 of the disclosure which comprises an inflatable balloon element 60 affixed at its proximal end to the distal end of an outer tubular body 20 and affixed at its distal end to the distal end of an inner tubular body 30. The annular lumen 22 between the outer tubular body 20 and inner tubular body 30 forms an inflation lumen for the inflatable balloon element 60 and is sized and adapted to contain the inflatable balloon element 60 in its invaginated configuration while maintaining a sufficiently open cross-section to allow the withdrawal of a fluid inflation medium such as sterile water or saline.

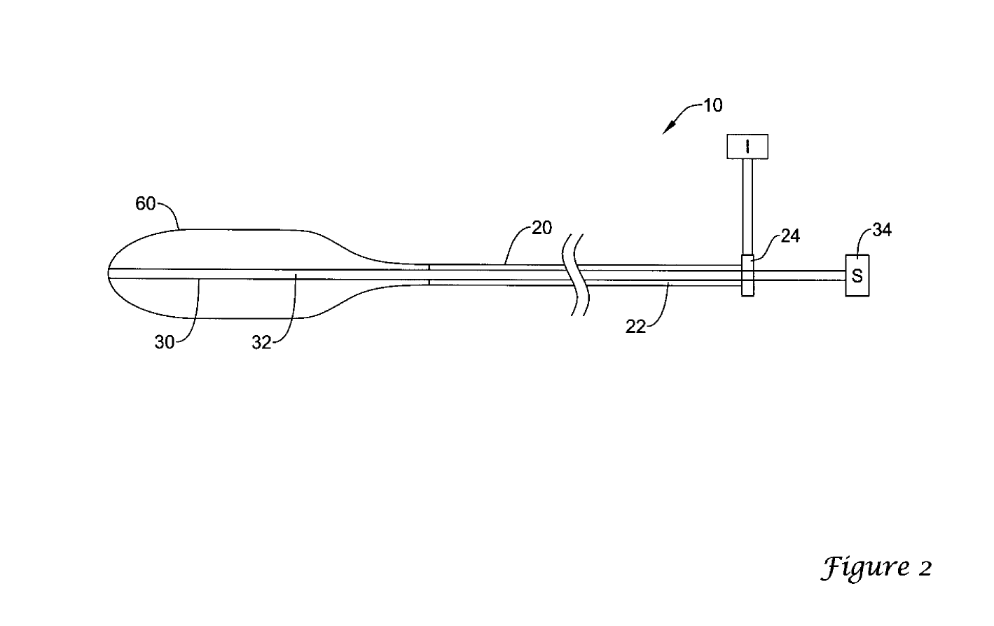

Although the outer tubular body 20 is depicted in FIG. 1 and the corresponding cross-sectional view of FIG. 2 as having a uniform and circular cross-sectional profile, it will be appreciated that the distal portion of outer tubular body 20 may, in some embodiments (not shown), be of somewhat larger cross-sectional area to better accommodate the invaginated inflatable balloon element 60 and may have an alternate cross-sectional shape. In other embodiments, substantially the entire length of the outer tubular body 20 may have a larger cross-sectional area than that illustrated schematically in FIGS. 1 and 2. In yet other embodiments, the outer tubular body 20 may include one or more separate inflation lumens and/or channels (not shown) which ensure that fluid inflation medium may freely enter and leave the inflatable balloon element 60 in all configurations which the invaginating inflatable balloon element 60 may assume as it is withdrawn.

The inflatable balloon element 60 may desirably be adapted to fold into a compact configuration as it invaginates first within itself and then within the outer tubular body 20 as inner tubular body 30 is withdrawn. The material of the inflatable balloon element 60 may be any of those commonly employed for the purpose in the art. In some embodiments, the outer surface of the inflatable balloon element 60 may include features such as ribs or protrusions which may facilitate breakup of plaque as the inflatable balloon element 60 expands during the valvuloplasty. In some embodiments, the inflatable balloon element 60 may have a non-circular cross-section to better orient the inflatable balloon element 60 within the valve to be dilated and/or to impart greater or lesser pressure to selected portions of the valve to be dilated. For example, an inflatable balloon element 60 for use in dilation of a tricuspid valve may have a somewhat triangular cross-section.

Although the distal and proximal ends of inflatable balloon element 60 have been depicted as being formed substantially continuously with one or both of outer tubular body 20 and inner tubular body 30, it will be appreciated that the ends of inflatable balloon element 60 may be formed from separate tapered elements and/or attachment portions which may fit over or within the inner and outer tubular bodies. In addition, the inflatable balloon element 60 may include one or more radially enlarged regions (not shown) which when inflated serve to position the inflatable balloon element 60 within the valve to be dilated during the dilation phase of the valvuloplasty procedure.

The proximal end region of outer tubular member 20 may include an adapter 24, such as a Touhy-Borst adapter or the like, illustrated schematically at the proximal end of the outer tubular member 20 of FIGS. 1 and 2 to sealingly and slidingly connect the outer tubular member 20 to the inner tubular body 30. In some embodiments, the adapter 24 may include an inflation port connecting the annular lumen 22, located between outer tubular member 20 and the inner tubular body 30, to a schematically indicated source “I” of fluid inflation medium. In other embodiments (not shown), the inflation port may be provided separately. The length of inner tubular member 30 is at least as long as, or somewhat longer than, the combined length of the outer tubular body 20 and the uninflated state of the inflatable balloon element 60 to allow the inflatable balloon element 60 to be fully extended distal of the distal end of the outer tubular body 20 in a first low profile delivery configuration.

The proximal end region of inner tubular member 30 may include an adapter 34, such as a Touhy-Borst adapter or the like, illustrated schematically at the proximal end of the inner tubular member 30 of FIGS. 1 and 2 to sealingly and slidingly connect the inner tubular member 30 to a guidewire 200 while the invaginating valvuloplasty balloon catheter is advanced through the patient's vasculature and to seal the proximal end of the lumen following removal of the guidewire 200. In some embodiments, the adapter 34 may include an aspiration port connecting the annular lumen guidewire/aspiration lumen 32, located within inner tubular member 30 to a schematically indicated aspiration source “S”. In other embodiments (not shown), the aspiration port may be provided separately.

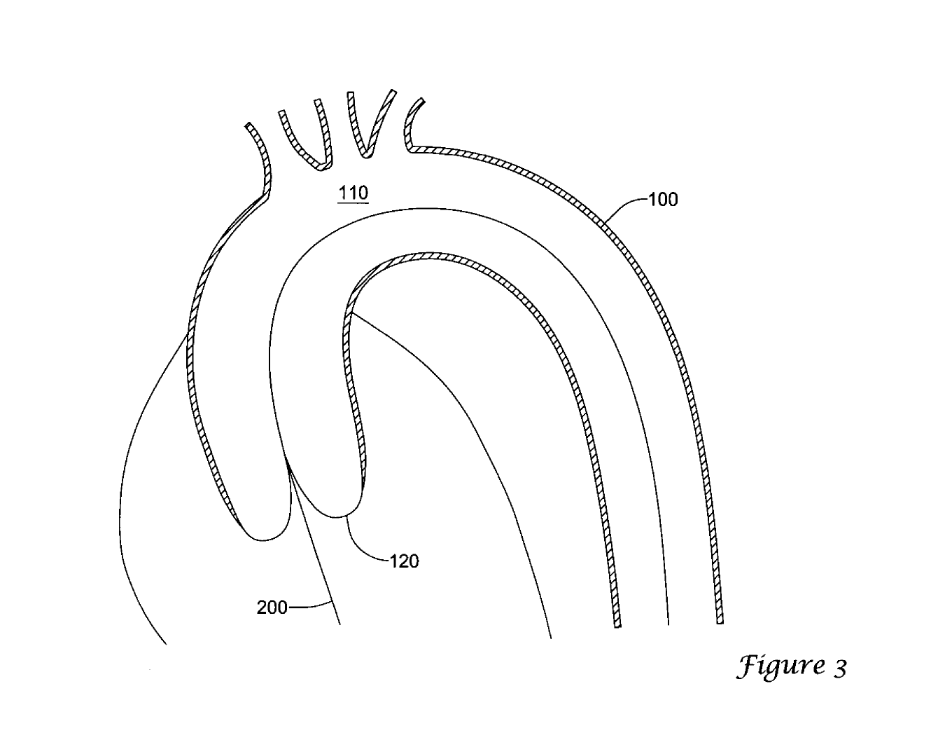

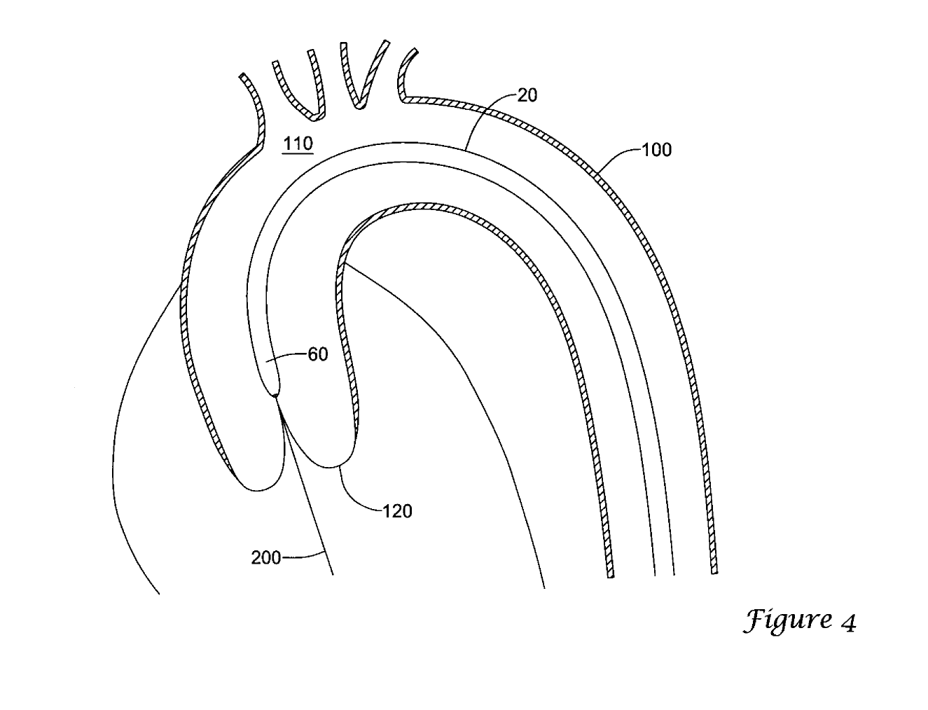

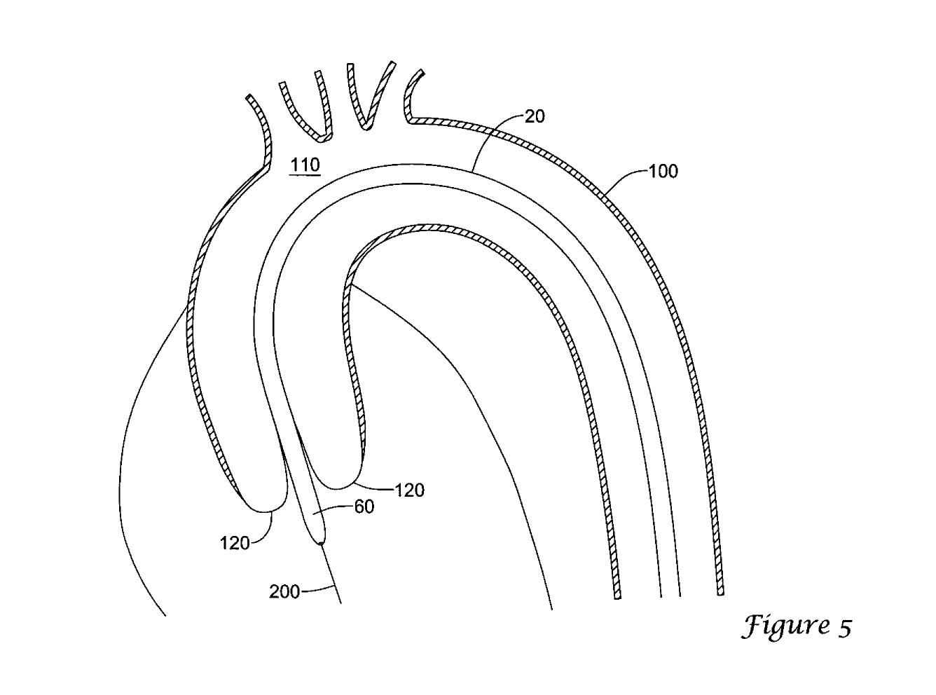

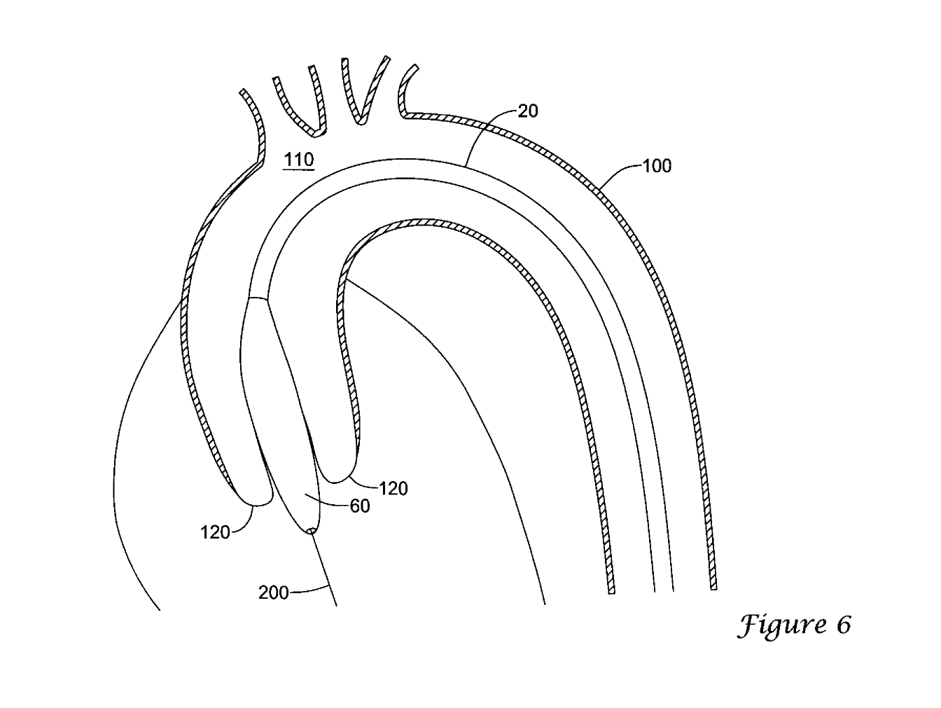

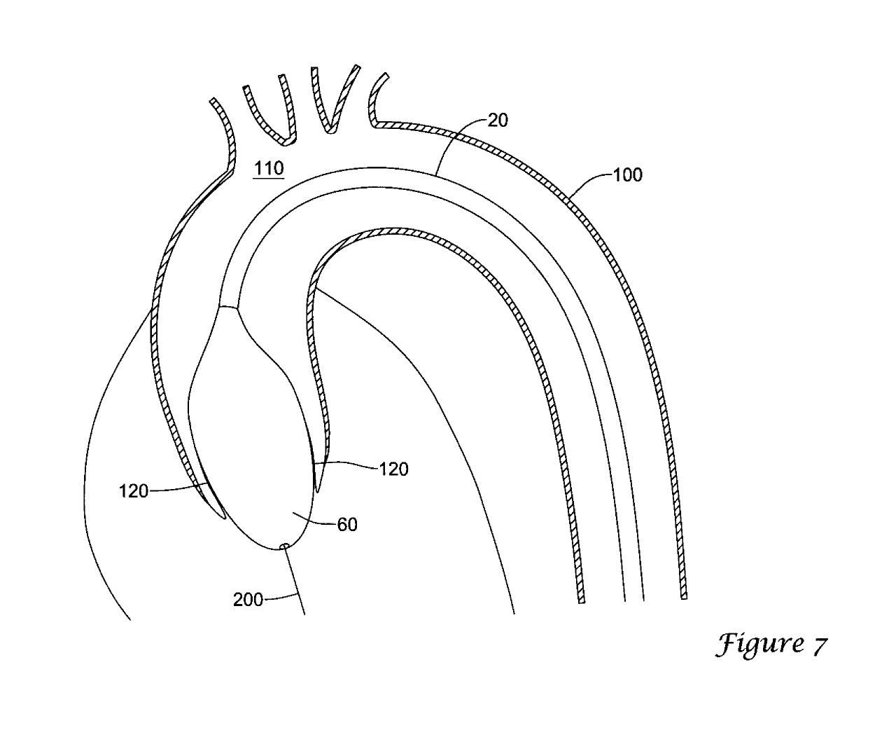

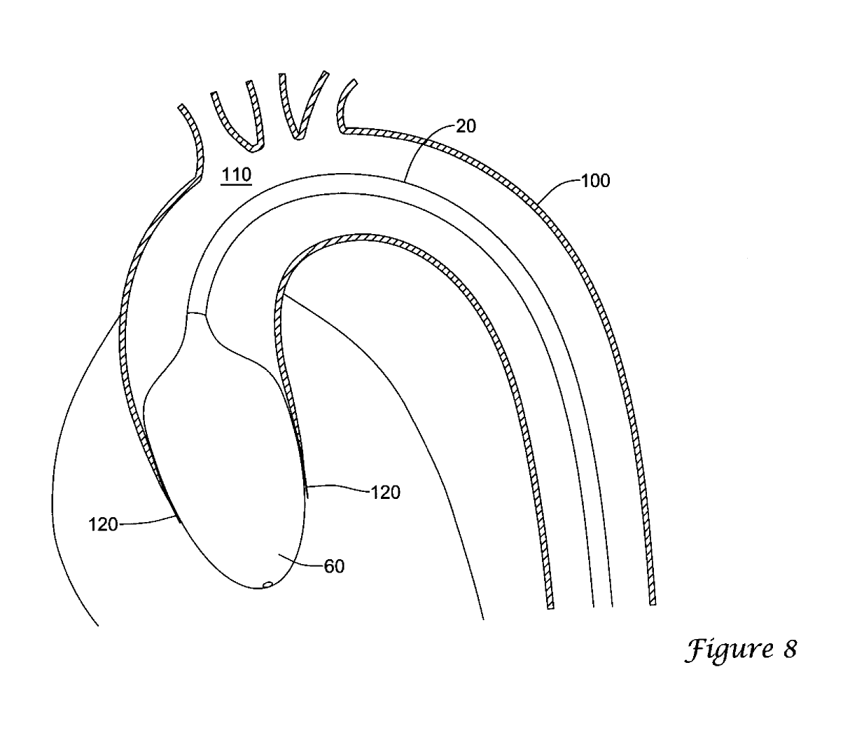

Use of the exemplary invaginating valvuloplasty balloon catheter 10 of the disclosure is illustrated in FIGS. 3-11. As shown in FIG. 3, a guidewire 200 is inserted transcutaneously and intravascularly in the normal manner and advanced through, for example, lumen 110 of the aorta 100, to a position slightly beyond the valve leaflets 120 or other tissue to be dilated. Guidewire 200 resides within the guidewire/aspiration lumen 32 of inner tubular body 30 as the invaginating valvuloplasty balloon catheter 10 is advanced to a position in which the inflatable balloon element 60 is adjacent the valve to be dilated (FIG. 4) and further advanced to within the valve (FIG. 5) in which the inflatable balloon 60 assumes a fully extended uninflated first configuration in which the inflatable balloon 60 lies distal of the distal end of the outer tubular body 20. The inflatable balloon 60 is then inflated as illustrated in FIGS. 6-8 by the introduction of inflation medium through an inflation port at the proximal end of outer tubular body 20 such that the inflated balloon 60 lies distal of the distal end of the outer tubular body 20 in a second configuration. As noted earlier, the inflation port may be a component of sealing adapter 24 or may directly connect source “I” of inflation medium to the annular inflation lumen 22.

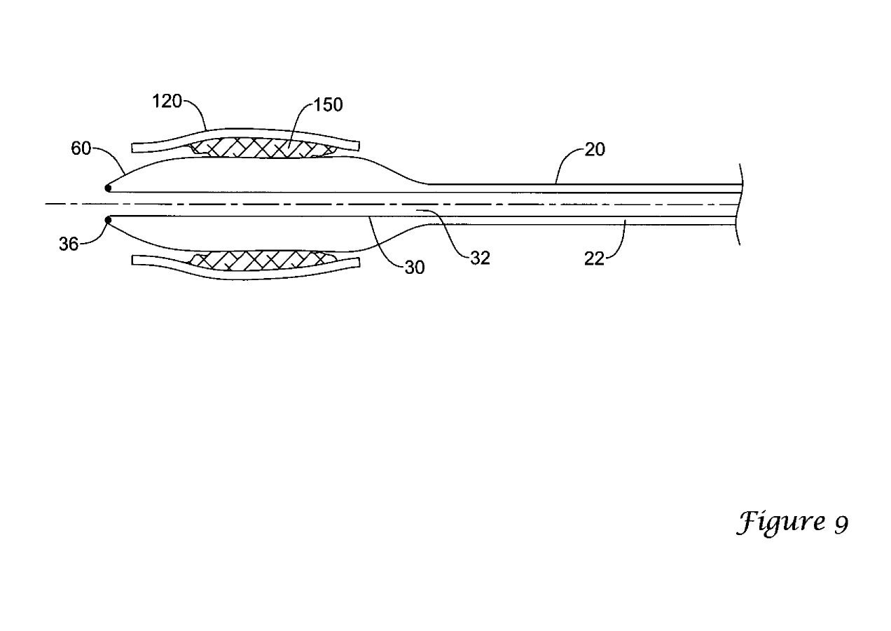

Although the guidewire 200 may be removed earlier in the procedure than illustrated in the inflation sequence, guidewire 200 may optionally be left in place during inflation such that it contributes to positional stability of the inflating balloon element 60. The invaginating valvuloplasty balloon catheter may include one or radiopaque and/or MRI visible markers 36 to assist positioning of the catheter. Once the inflatable balloon element 60 has been fully inflated (FIG. 9) to compress and disrupt the plaque associated with the valve leaflets 120 to be dilated, possibly with cycling of the inflation pressure, and the guidewire 200 has been removed, aspiration is applied to guidewire/aspiration lumen 32 through an aspiration port at the proximal end of inner tubular body 30, which port may be a component of sealing adapter 34 or which may directly connect aspiration source ‘S” to aspiration lumen 32.

As used herein aspiration may be passive or active. Passive aspiration uses the native blood pressure to move fluid and debris through inner tubular body 30 to a point outside the body. The use of passive aspiration requires that pressure within the aspiration lumen 32 be lower than the peak systolic blood pressure. Should the aspiration lumen 32 become clogged by debris or clot, more aggressive aspiration may involve the use of an active aspiration mechanism. The active aspiration mechanism may be, for example, an aspiration syringe or an aspiration pump and the aspiration may be applied in either a continuous or intermittent manner. If aspiration volumes are significant, the aspirated blood/debris can be filtered and returned to the body via, for example, a femoral access sheath inserted on either side of the body.

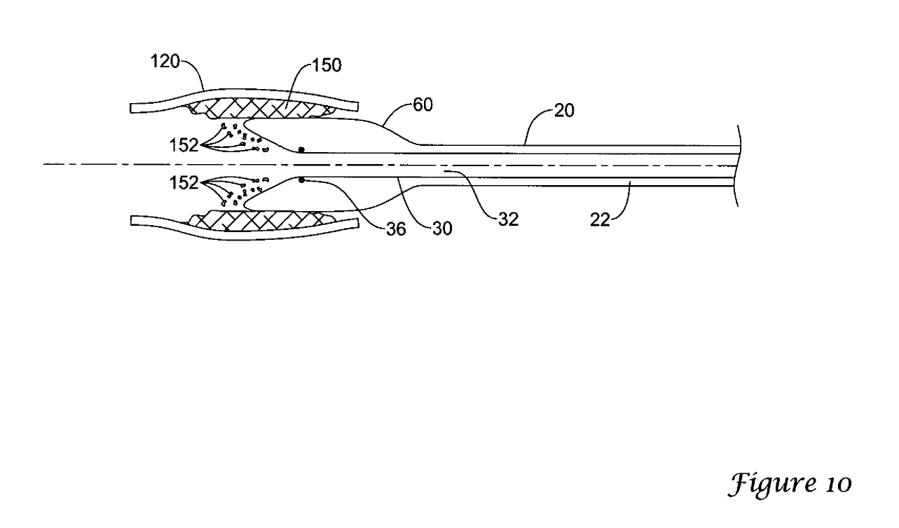

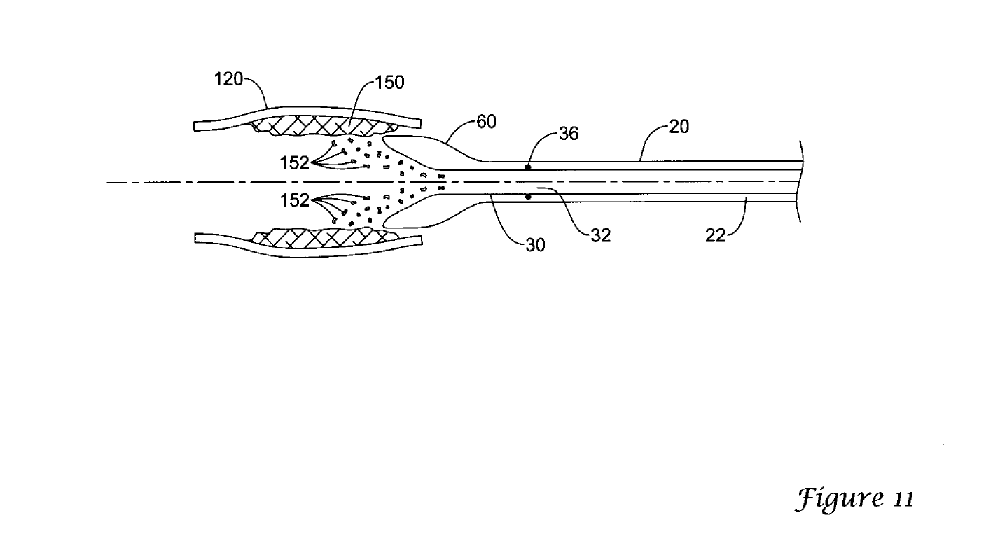

While continuing to aspirate through aspiration lumen 32, inner tubular body 30 may be withdrawn proximally relative to outer tubular body 20 with simultaneous or staged withdrawal of inflation medium from the inflatable balloon element 60 which allows the inflatable balloon element 60 to partially collapse and roll back within itself in a third configuration illustrated in FIG. 10 and then within the outer tubular body 20 without displacing that portion of the inflatable balloon 60 which is in contact with the dilated valve leaflets 120 and/or plaque 150 to be displaced. As the inflatable balloon element 60 invaginates and withdraws from the dilated valve leaflets 120, the applied aspiration gathers and removes debris 152 from the at least partially disrupted plaque 150 allowing it to be removed through lumen 32 of inner tubular body 30 as illustrated in FIGS. 10 and 11.

Removal of inflation medium from inflatable balloon element 60 with attendant withdrawal of inner tubular body 30 relative to outer tubular body 20 while continuing to apply aspiration to aspiration lumen 32 of inner tubular body 30 may continue until the invaginating inflatable balloon 60 is 50%, 75%, or even 100% invaginated within outer tubular body 30 to form a fourth configuration of the invaginating valvuloplasty catheter 10 in which at least half of the inflatable balloon element lies proximal of the distal end of the outer tubular body 20. Once the invaginating inflatable balloon 60 has been released from the dilated valve leaflets 120, the valvuloplasty is essentially complete; the inflatable balloon 60 may be deflated and optionally may be fully invaginated within outer tubular body 20; and the invaginating valvuloplasty balloon catheter 10 may be removed from the patient's body.

Although the illustrative example described above relate to an invaginating inflatable balloon catheter adapted primarily for valvuloplasty, other uses are also contemplated. In such an embodiment, the size of the inflatable invaginating balloon may be altered to better suit the vessel or organ to be dilated and the lengths of the respective inner and outer tubular bodies may be altered to more conveniently reach the dilation site.

Various modifications and alterations of the device of this disclosure will become apparent to those skilled in the art without departing from the scope and principles of the device disclosed herein, and it should be understood that the invention is defined by the claims and is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. An invaginating valvuloplasty balloon catheter comprising:
an outer tubular body having a distal end and a proximal end;
an inner tubular body having a distal end and a proximal end defining a lumen therebetween adapted to receive guidewire and together with the outer tubular body defining an annular inflation lumen therebetween; and
an inflatable balloon element having a distal end, a proximal end an uninflated state and an inflated state,
wherein the distal end of the inflatable balloon element is sealingly attached to distal end of the inner tubular body and the proximal end of the inflatable balloon element is sealingly attached to the outer body;
further wherein the outer body is sized and adapted to contain the inflatable balloon element in an invaginated configuration while maintaining a fluid connection between a source of inflation medium at the proximal end thereof and the proximal end of the inner tubular body is adapted to connect with a source of aspiration.

2. The invaginating valvuloplasty balloon catheter of claim 1, wherein the inner tubular body has a length greater than the combined length of the outer tubular and the inflatable balloon element in the uninflated state thereof.

3. The invaginating valvuloplasty balloon catheter of claim 1, wherein the outer tubular body includes an inflation medium port proximate the proximal end of the outer tubular body.

4. The invaginating valvuloplasty balloon catheter of claim 1, wherein the outer tubular body includes an adapter adapted to sealingly and slidingly connect the proximal end of the outer tube to a proximal region of the inner tubular body.

5. The invaginating valvuloplasty balloon catheter of claim 1, wherein the proximal end of the inner tubular body adapted to connect with an aspiration source includes an aspiration port proximate the proximal end of the outer tubular body.

6. The invaginating valvuloplasty balloon catheter of claim 1, wherein the inner tubular body includes an adapter adapted to sealingly and slidingly connect the proximal end of the inner tube to a guidewire.

7. The invaginating valvuloplasty balloon catheter of claim 1, wherein the inflatable balloon element has an uninflated first configuration in which the inflatable balloon element lies distal of the distal end of the outer tubular body.

8. The invaginating valvuloplasty balloon catheter of claim 1, wherein the inflatable balloon element has an inflated second configuration in which the inflatable balloon element lies distal of the distal end of the outer tubular body.

9. The invaginating valvuloplasty balloon catheter of claim 1, wherein the inflatable balloon element has a partially inflated third configuration in which at least a portion of the inflatable balloon element lies proximal of the distal end of the outer tubular body and within the outer tubular member.

10. The invaginating valvuloplasty balloon catheter of claim 1, wherein the inflatable balloon element has a partially inflated fourth configuration in which more than half of the inflatable balloon element lies proximal of the distal end of the outer tubular body and within the outer tubular member.

11. A method of performing a valvuloplasty comprising:
inserting a guidewire through a patient's vasculature;
inserting the guidewire through the valve to be dilated;
advancing an invaginating valvuloplasty balloon catheter through the patient's vasculature;
advancing a balloon element of an invaginating valvuloplasty balloon catheter at least partially through the valve to be dilated;
inflating the balloon element of the invaginating valvuloplasty balloon catheter by introducing an inflation medium through a proximal inflation port associated with an outer tubular body of the invaginating valvuloplasty balloon catheter thereby at least partially inflating the balloon and dilating the valve to be dilated;
removing the guidewire from the invaginating valvuloplasty balloon catheter;
applying aspiration to an aspiration lumen associated with inner tubular body while partially removing at least a portion of the inflation medium from the balloon element and withdrawing the inner tubular body proximally through the outer tubular body thereby removing debris dislodged by dilation of the valve to be dilated;
deflating the balloon element by removing the inflation medium; and
removing the invaginating valvuloplasty balloon catheter from the patient's body.

12. The method of performing a valvuloplasty of claim 11, wherein the inner tubular body has a length greater than the combined length of the outer tubular body and the inflatable balloon element in the uninflated state thereof.

13. The method of performing a valvuloplasty of claim 11, wherein the outer body is sized and adapted to contain the inflatable balloon element in an invaginated configuration while maintaining a fluid connection between a source of inflation medium at the proximal end thereof.

14. The method of performing a valvuloplasty of claim 11, wherein the proximal end of the outer tubular body is adapted to sealingly and slidingly connect the proximal end of the outer tube to a proximal region of the inner tubular body.

15. The method of performing a valvuloplasty of claim 11, wherein inner tubular body includes an adapter adapted to sealingly and slidingly connect the proximal end of the inner tube to a guidewire.

16. The method of performing a valvuloplasty of claim 11, wherein inflating the balloon element of the invaginating valvuloplasty balloon catheter by introducing an inflation medium includes one or more cycles of inflation and partial deflation to disrupt plaque which may be present.

17. The method of performing a valvuloplasty of claim 11, wherein the inflation medium is saline.

18. The method of performing a valvuloplasty of claim 11, wherein the inflation medium is sterile water.

19. The method of performing a valvuloplasty of claim 11, wherein the step of applying aspiration to an aspiration lumen associated with the inner tubular body while partially removing at least a portion of the inflation medium from the balloon element and withdrawing the inner tubular body proximally through the outer tubular body continues until at least half of the inflatable balloon element lies proximal of the distal end of the outer tubular body.

20. The method of performing a valvuloplasty of claim 11, wherein the step of applying aspiration to an aspiration lumen associated with the inner tubular body while partially removing at least a portion of the inflation medium from the balloon element and withdrawing the inner tubular body proximally through the outer tubular body continues until at least three quarters of the inflatable balloon element lies proximal of the distal end of the outer tubular body.
